(19) United States
(12) Patent Application Publication
(10) Pub. No.: US 2025/0376725 A1
(43) Pub. Date: Dec. 11, 2025

(54) NANOPORE-BASED SCANNING SYSTEM AND METHOD

(71) Applicant: ECOLE POLYTECHNIQUE FEDERALE DE LAUSANNE (EPFL), Lausanne (CH)

(72) Inventors: Aleksandra RADENOVIC, ECUBLENS VD (CH); Georg E. FANTNER, CHAVANNES-RENENS (CH); Samuel LEITÃO, LAUSANNE (CH); Vytautas NAVIKAS, ST-SULPICE VD (CH)

(21) Appl. No.: 18/869,737

(22) PCT Filed: Jun. 1, 2023
(86) PCT No.: PCT/IB2023/055626

§ 371 Date: Nov. 26, 2024

(30) Foreign Application Priority Data
Jun. 1, 2022 (WO) PCT/IB2022/055136

(51) Int. Cl. C12Q 1/6869; G01N 21/64; G01N 33/487

(52) U.S. Cl. CPC C12Q 1/6869; G01N 21/6428; G01N 33/48721

(57) ABSTRACT

Nanopore-based scanning system including a probe structure comprising a nanopore: suction means configured to draw an end of a (bio)molecule inside the nanopore and inside the probe structure, single or multiple times; and displacement means configured to mechanically displace the probe structure and the nanopore relative to the one (bio)molecule along a direction following a direction of extension of the (bio)molecule while the (bio)molecule is located inside the nanopore and inside the probe structure, or configured to mechanically displace at least one support holding the (bio)molecule relative to the nanopore along a direction following a direction of extension of the (bio)molecule while holding the at least one support and while the (bio)molecule is located inside the nanopore.

### CROSS-REFERENCE TO RELATED APPLICATIONS

The present application claims priority to international patent application PCT/IB2022/055136 filed on Jun. 1, 2022, the entire contents thereof being herewith incorporated by reference.

### FIELD OF THE INVENTION

The present invention generally belongs to the fields of molecular analysis and diagnostics. In particular, the invention pertains to methods and systems for controlled translocation of molecules, such as polymeric molecules, through nanopore-based devices.

### BACKGROUND

Nanopores have emerged as a label-free single molecule (DNA, RNA, peptide, protein, polymers, glycans) sensing tool based on ionic-current variations, translocating single molecules through a small opening. Nanopore-based approaches such as solid-state nanopores, biologic-nanopores, and glass nanopores are label free methods that operate in attomolar conditions, well suited to reveal variety of the single molecule properties.

The main challenge in nanopores is the uncontrolled dynamics of the free-translocation characterized by a non-linear velocity, dependent on the charge of the molecule and the bias. Ultimately, fast speeds of free-translocations limit temporal and spatial resolution due to a finite amplifier bandwidth. This limitation decreases the signal-to-noise ratio (SNR), which is critical to detecting the single molecule topologies and/or sequence. The high translocation speeds and low SNR of free translocations have prevented solid-state devices to be used as a robust tool for DNA sequencing. Biological nanopores systems, on the other hand, are successfully used for DNA sequencing, however at the moment, they can only detect ssDNA due to their molecular designs.

Glass nanopores in the form of nanocapillaries are an alternative to translocate single molecules, and have been demonstrated to be a robust and cost-effective platform that uses a glass nanopore for detecting various analytes. Glass nanopores can be manufactured to radii below 10 nm by precisely controlling the diameter of the opening, for example through electron beam irradiation or by depositing the additional coatings by controllable wet-chemical silanization. Furthermore, glass nanopores demonstrate good SNR characteristics for high-bandwidth measurements, due to a low capacitance of silicate reducing high-frequency noise. However, the challenge remains the same, to control the dynamics of the translocation.

Scanning ion conductance microscopy (SICM) uses nanocapillaries as a probe to image surfaces by moving a glass nanopore with picometer precision towards a surface while measuring the current to detect distance between the nanopore and the surface. This microscopy method reveals nanostructures on the cell membrane, and can be combined with optical microscopy such as super-resolution fluorescence techniques. However, SICM has never been suggested and adapted for nanopore-based translocation of single molecules.

There is still a need for rapid and cheap molecular (e.g. DNA, peptide, protein or RNA) sequencing technologies across a wide range of applications. Existing technologies are slow and expensive, mainly because they rely on amplification techniques to produce large volumes of nucleic acid and require a high quantity of specialist fluorescent chemicals for signal detection. Nanopore sensing has the potential to provide rapid and cheap molecular sequencing by reducing the quantity of molecules and reagents required. However, the available nanopore-based solutions for molecular sequencing are still far to be optimized, particularly when it comes to control the dynamics of the translocation.

In relation to the prior art: Keyser et al. (Nature Physics 2.7 (2006):) and Trepagnier et al. (Nano Lett. 2007 September; 7(9):2824-30) described a method and a system for controlling DNA capture and propagation through artificial nanopores. λ-DNAs were connected to 10 μm polystyrene beads via streptavidin-biotin linkage. The optically trapped beads were trapped and held in proximity to a single artificial nanopore in a membrane separating two chambers. Electrodes maintained an electrical potential across the nanopore. The bead is pulled out of the trap and towards the pore membrane due to the capture and electric-field-driven translocation of DNA through the pore. The optical tweezers allows to trap a sample above a pore or channel, reduce polymer propagation speeds, and repeatedly carry out measurements on one DNA molecule. However, the random positioning of beads with regards to the nanopores, the removal of the bead from the optical trap and the aleatory contact of biomolecules with the nanopores for analysis results in an insufficient signal-to-noise ratio for robust DNA sequencing and an unsatisfactory control of the dynamics of the translocation, and renders this technique unsuitable for a systematic investigation of biomolecules, particularly in case of rare or precious samples.

### SUMMARY

It is therefore one aspect of the present disclosure to provide a nanopore scanning system or a nanopore-based scanning system according to claim 1 that addresses the above-mentioned inconveniences and needs.

Another aspect of the present disclosure concerns a nanopore scanning method or a nanopore-based scanning method according to claim 27 or claim 28 that also addresses the above-mentioned inconveniences and needs.

Further advantageous features are provided in the dependent claims.

In order to address and overcome at least some of the above-mentioned drawbacks of the prior art solutions, the present inventors developed a new approach for controlling the translocation of molecules or biomolecules ((bio)molecules), particularly polymeric (bio)molecules, through nanopore devices and systems, having improved features and capabilities.

In particular, the main purpose of the present invention is that of providing a platform to rapidly and robustly allow controlled translocation of (bio)molecules, particularly polymeric molecules such as glycans, oligo/polypeptides or nucleic acids through solid state nanopores, for an unparalleled precision in detecting, for instance, complex topological variations in DNA.

This aim has been accomplished with the present invention, as described herein and in the appended claims.

The inventors proved able to implement a nanopore-based scanning probe approach named scanning ion conductance spectroscopy (SICS), based on, for example, a modified scanning ion conductance microscopy SICM platform to perform controlled-translocations according to one non-limiting and exemplary embodiment. The present system and method overcome critical limitations in nanopore technology, by, for example, enabling a controlled translocation with constant velocity and averaging many readings on the same molecule in a deterministic fashion. In embodiments of the invention, it allows, for example, the mapping of thousands of DNA molecules tethered, in one embodiment, on a glass surface and allows for example a combination with fluorescence microscopy methods (FIG. 1a). The invention is not limited to DNA but can adapted to any analyte for which linking strategy to a surface exist, for example through linking to DNA handles (RNA, peptides, glycans, artificial polymers etc).

The method and system of the invention overcome the main challenge in current nanopore systems—the uncontrolled dynamics of the translocation. A glass nanopore was used, for example, as a scanning probe to deterministically translocate and map out, for example, molecules tethered on a glass surface at a constant velocity, to perform controlled-translocations independent of the applied voltage bias, salt concentration, and pH. The method of the invention was successfully applied to molecular rulers, DNA gaps, Hairpins, and DNA-dCas9 complexes with correlative fluorescence imaging.

### Additional Advantages of the Method and System of the Invention are the Following:

the method enables high-throughput data acquisition above 100′000 readings per experiment and an adjustable scanning rate of 4-500 readings/s;
conductance-distance curves with SICS increases the SNR by 2 orders of magnitude compared to free-translocations, by decreasing the velocity with a constant motion and by averaging multiple readings of the same molecule;
detection of nanostructures can be detected along DNA strands with a location precision above 99% showed by custom-designed DNA rulers and detected amplitudes on custom-designed DNA gaps (80-, 40-, 20- and 12-nucleotides) with an error below 0.003%;
the method and system of the invention allow single-base detection capabilities in dsDNA. Such detectability on dsDNA with nanopores has never been reported before according to the inventors knowledge;
the method and system of the invention can be combined with fluorescence microscopy methods for additional information. In addition, it is compatible with micro- and nano-arrays of (bio)molecules pattering.

Methods and systems according to the invention are promising platforms for the development of diverse nanopore-based probes compatible with solid-state and biological nanopore systems. The scanning platform of the invention allows micro- and nano-patterning configuration on a glass substrate, and can allow to perform automatic screening of molecular arrays, for example DNA arrays) for more capable point-of-care diagnostic devices. Additionally, the methods and systems according to the invention are suitable for operation with other kind of charged (bio)molecules, particularly biopolymers, including (oligo) peptides, proteins, denaturated proteins, aptamers, hybrid nucleic acid/peptide constructs and the like. Furthermore, the methods and systems according to the invention are suitable for detection and analysis of any changes that modify size/structure or the charge of the biomolecule. Examples of such editing include for example: binding of the molecules/conformational changes or post-translational modifications (PTMs) of charged biopolymers, particularly (oligo) peptides, proteins and hybrid nucleic acid/peptide constructs.

In view of the above-summarized drawbacks and/or problems affecting devices of the prior art, according to the present invention there is provided a nanopore-based scanning system according to claim 1.

Another object of the present invention relates to a nanopore-based scanning method according to claim 27 or 28. Further advantageous features can be found in the dependent claims.

Further embodiments of the present invention are defined by the appended claims.

The above and other objects, features and advantages of the herein presented subject-matter will become more apparent from a study of the following description with reference to the attached figures showing some preferred aspects of said subject-matter.

### BRIEF DESCRIPTION OF THE DRAWINGS

The accompanying drawings, which are incorporated herein and constitute part of this specification, illustrate the presently preferred embodiments of the invention, and together with the general description given above and the detailed description given below, serve to explain features of the invention.

FIGS. 1a to 1d show controlled-translocations of DNA with a nanopore-based scanning probe microscopy or system according to the present disclosure.

FIG. 1a is a schematic illustration of an exemplary nanopore-based scanning system or scanning ion conductance spectroscopy (SICS) system according to the present disclosure, optionally combined with fluorescence microscopy.

FIG. 1b shows the principle of SICS step-by-step that can be implemented by the nanopore-based scanning system or scanning ion conductance spectroscopy (SICS) system of the present disclosure. Step 1: DNA captured through the nanopore by an electrophoretic force generated by a positive bias (200-600 mV). Step 2: Translocation along the DNA length. Step 3: Identification of a feature. Step 4: Complete translocation followed unlimited translocation cycles in the same molecule or map out different molecules in, for example, a 100×100 μm² area. The generated data corresponds to a conductance-distance curve revealing the DNA nanostructure, identifying a DNA-dCas9 complex in this exemplary case (curve CV). A 20 nm glass nanopore radius at 1 μm/s translocation velocity was used in this exemplary embodiment, with a 200 mV bias in 400 mM KCl, PH=7.4.

FIG. 1c shows a Free-translocation signature of a DNA ruler with an exemplary 8 nm glass nanopore radius in exemplary optimal conditions, 600 mV bias in 4 M LiCl, pH=7.4.

FIG. 1d shows a SICS controlled-translocation signature, obtained using the nanopore-based scanning system of the present disclosure, of a DNA ruler in exemplary optimal conditions with an exemplary 8 nm glass nanopore radius at 1 μm/s translocation velocity, 300 mV bias in 1 M KCl, pH=7.4. The DNA molecules are tethered to a glass surface, for example, via Biotin-Streptavidin biding.

FIGS. 2a to 2g show controlled-translocations of custom-designed DNA rulers.

FIG. 2a shows a design of an. exemplary DNA ruler construct, composed of 7′228 base pairs with 6 markers containing DNA dumbbell hairpins separated by equal 1032 base pair (bp) intervals.

FIG. 2b shows controlled-translocation of the exemplary DNA ruler using for example a 7 nm radius glass nanopore at 1 μm/s translocation velocity, and translocation averaged to 1 nm.

FIG. 2c shows controlled-translocation at several velocities: 0.1 μm/s, 1 μm/s, 10 μm/s and 100 μm/s; for an exemplary 8 nm radius glass nanopore.

FIG. 2d shows the signal-to-noise ratio SNR of the detected marker (1st from the free-end) at different translocation velocities (n=10).

FIG. 2e shows the distribution of the detected marker amplitude (1st from the free-end) and distance between markers (1st and 2nd from the free-end), in 1 μm/s controlled-translocations vs free-translocations, with glass nanopores of similar size (8 nm radius), centred to the average amplitude (μ1) and average ΔG_{distance} (μ2), n=100.

FIG. 2f shows a controlled-translocation overlay of 100 different molecules tethered on the surface at 1 μm/s translocation velocity. Molecules were aligned to the DNA exit.

FIG. 2g shows a controlled-translocation overlay of the same molecule 100 times and average (in white) using an exemplary 8 nm radius glass nanopore at 1 μm/s translocation velocity. All controlled-translocation experiments were performed with 300 mV bias in 1 M KCl, pH=7.4.

FIGS. 3a to 3f show controlled-translocations of custom-designed DNA gaps.

FIG. 3a shows an exemplary design of a DNA gap construct composed of 9′276 base pairs (bp), containing a gap in the middle of 4 different sizes: 80-, 40-, 20- and 12-nucleotides.

FIG. 3b Bottom shows a controlled-translocation of 80-nucleotides DNA gap, with a conductance amplitude (ΔG_{gap}) of half the dsDNA translocation amplitude (ΔG_{dsDNA}); using an exemplary 12 nm glass nanopore radius at 1 μm/s translocation velocity and translocation averaged to 1 nm. FIG. 3b Top shows a probability density map of 1000 readings from the same 80 bases DNA gap; with an exemplary 10 nm glass nanopore radius at 1 μm/s translocation velocity, and translocation averaged to 1 nm. Acquired at 4 readings/s.

FIG. 3c is a probability density map of bidirectional translocations on 80- and 12-nucleotides DNA gap with the same pipette (n=100); for an exemplary 12 nm glass nanopore radius at 1 μm/s translocation velocity, and translocation averaged to 0.01 nm.

FIG. 3d shows controlled-translocation signals overlay of 80-, 40-, 20-, and 12-nucleotides and corresponding waterfall plot (n=10 with the average in dark color). An exemplary 8 nm glass nanopore radius was used at 1 μm/s translocation velocity.

FIG. 3e shows the amplitude of the detected gaps (ΔG_{gap}) vs the number of nucleotides with different pipettes.

FIG. 3f shows the detection of a 1 base nick with an exemplary 8 nm glass nanopore radius at 1 μm/s translocation velocity and translocation averaged to 1 nm. A complimentary 19 bases oligonucleotide was hybridized on a 20 bases gap. All the experiments were performed with 300 mV bias in 1 M KCl, pH=7.4.

FIG. 4 shows a non-limiting and exemplary DNA peptide construct that may be sensed and/or sequenced by the system and method of the present disclosure.

FIGS. 5a and 5b show a non-limiting example of post-translational modifications monitoring that can be performed, for example on peptides, by the system and method of the present disclosure. The illustrated example involves the use of an exemplary ligand that is nitrilotriacetic acid (NTA) that can specifically bind to nickel ions.

Herein, identical reference numerals are used, where possible, to designate identical elements that are common to the Figures. Also, the images are simplified for illustration purposes and may not be depicted to scale.

### DETAILED DESCRIPTION OF SEVERAL EMBODIMENTS

The subject-matter described in the following will be clarified by means of a description of those aspects which are depicted in the drawings. It is however to be understood that the scope of protection of the invention is not limited to those aspects described in the following and depicted in the drawings; to the contrary, the scope of protection of the invention is defined by the claims. Moreover, it is to be understood that the specific conditions or parameters described and/or shown in the following are not limiting of the scope of protection of the invention, and that the terminology used herein is for the purpose of describing particular aspects by way of example only and is not intended to be limiting.

Unless otherwise defined, technical and scientific terms used herein have the same meaning as commonly understood by one of ordinary skill in the art to which this invention belongs. Further, unless otherwise required by the context, singular terms shall include pluralities and plural terms shall include the singular. The methods and techniques of the present disclosure are generally performed according to conventional methods well known in the art and as described in various general and more specific references that are cited and discussed throughout the present specification unless otherwise indicated. Further, for the sake of clarity, the use of the term “about” is herein intended to encompass a variation of +/−10% of a given value.

Non-limiting aspects of the subject-matter of the present disclosure will be described by way of example with reference to the accompanying figures, which are schematic and are not intended to be drawn to scale. For purposes of clarity, not every component is labelled in every figure, nor is every component of each aspect of the invention shown where illustration is not necessary to allow those of ordinary skill in the art to understand the invention.

The following description will be better understood by means of the following definitions.

As used in the following and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where for the description of various embodiments use is made of the term “comprising”, those skilled in the art will understand that in some specific instances, an embodiment can be alternatively described using language “consisting essentially of” or “consisting of.”

In the frame of the present disclosure, the expression “operatively connected” and similar reflects a functional relationship between the several components of the device or a system among them, that is, the term means that the components are correlated in a way to perform a designated function. The “designated function” can change depending on the different components involved in the connection. Likewise, any two components capable of being associated can also be viewed as being “operably couplable,” to each other to achieve the desired functionality. A person skilled in the art would easily understand and figure out what are the designated functions of each and every component of the device or the system of the invention, as well as their correlations, on the basis of the present disclosure.

The term “nucleotide” refers to a molecule that contains a nitrogen-containing heterocyclic base (also referred to as “nucleobase”), a sugar or a modified sugar and one or more phosphate groups. For example, in some embodiments, a nucleotide can be a deoxynucleotide triphosphate (dNTP). The term “non-natural nucleotide” as used herein refers to a nucleotide that obeys Watson-Crick base pairing but has a modification that can be detected. By way of example, but not limitation, such a modification can be a functional group attached to the nucleobase such as a methyl group on methylcytosine.

As used herein, the terms “nucleic acid molecule,” “nucleic acid sequence,” “nucleic acid fragment,” “oligonucleotide” and “polynucleotide” are used interchangeably and refer to biopolymers that are made from nucleotides as monomer units. The nucleotide monomers link up to form a linear sequence of the nucleic acid polymer. Nucleic acids encompassed by the present disclosure can include deoxyribonucleic acid (DNA), ribonucleic acid (RNA), cDNA or a synthetic nucleic acid known in the art, such as glycerol nucleic acid (GNA), threose nucleic acid (TNA), locked nucleic acid (LNA) or other synthetic polymers with nucleotide side chains, or any combination thereof. For the sake of easiness, peptide nucleic acids (PNAs), artificially synthesized polymer similar to DNA or RNA, are also included into the definition of oligonucleotides according to the invention.

Nucleotide subunits of nucleic acids can be naturally occurring, artificial, or modified. As indicated above, nucleotide typically contains a nucleobase, a sugar, and at least one phosphate group. The nucleobase is typically heterocyclic. Suitable nucleobases include the canonical purines and pyrimidines, and more specifically adenine (A), guanine (G), thymine (T) (or typically in RNA, uracil (U) instead of thymine (T)), and cytosine (C). The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate or triphosphate. These are generally referred to herein as nucleotides or nucleotide residues to indicate the subunit. Without specific identification, the term nucleotides, nucleotide residues, and the like, is not intended to imply any specific structure or identity.

As indicated above, the nucleic acids of the present disclosure can also include synthetic variants of DNA or RNA. “Synthetic variants” encompasses nucleic acids incorporating known analogs of natural nucleotides/nucleobases that e.g. can hybridize to nucleic acids in a manner similar to naturally occurring nucleotides. Exemplary synthetic variants include peptide nucleic acids (PNAs), phosphorothioate DNA, locked nucleic acids, and the like. Modified or synthetic nucleobases and analogs can include, but are not limited to, 5-Br-UTP, 5-Br-dUTP, 5-F-UTP, 5-F-dUTP, 5-propynyl dCTP, 5-propynyl-dUTP, diaminopurine, S2T, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N 6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-Dmannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Persons of ordinary skill in the art can readily determine what base pairings for each modified nucleobase are deemed a base-pair match versus a base-pair mismatch.

Related to the detection of the any changes in biomolecule (editing) such as the modification of its conformation, its size, its charge we give here specific example. The invention further provides a method for determining the presence, absence, number and/or position(s) of one or more post-translational modifications (PTMs) in a peptide, polypeptide, protein or hybrid nucleic acid/peptide constructs. The peptide, polypeptide, protein or hybrid nucleic acid/peptide constructs is contacted with a nanopore according to the invention such that the peptide, polypeptide, protein or hybrid nucleic acid/peptide constructs moves through the pore. One or more current measurements are taken as the target analyte moves with respect to the pore, thereby determining of the presence, absence, number and/or position(s) of one or more PTMs in the target analyte.

The method of the invention allows the rapid detection of PTMs at the single-molecule level through alterations in the current signature through the pore. The method of the invention has several advantages over conventional methods for studying PTMs. It is rapid and simple. It is sensitive because it can identify single PTMs and as well as multiple PTMs. It can also distinguish between adjacent PTMs. The output from the method is analysed in real time, allowing it to be stopped when sufficient information has been obtained. The method can be carried out by someone with minimal training or qualification. The presence or absence of PTMs, such as phosphorylations, may be used to diagnose diseases.

The method of the invention also allows the number and position(s) of one or more specific PTMs to be determined. The position(s) of the PTMs refers to their/its position(s) in the peptide, polypeptide, protein or hybrid nucleic acid/peptide construct, such as the PTM site or the amino acid which is modified.

PTMs are preferably selected from modification with a hydrophobic group, modification with a cofactor, addition of a chemical group, glycation (the non-enzymatic attachment of a sugar), biotinylation and pegylation. PTMs can also be non-natural, such that they are chemical modifications done in the laboratory for biotechnological or biomedical purposes.

The modification with a hydrophobic group is preferably selected from myristoylation, attachment of myristate, a C14 saturated acid; palmitoylation, attachment of palmitate, a C16 saturated acid; isoprenylation or prenylation, the attachment of an isoprenoid group; farnesylation, the attachment of a farnesol group; geranylgeranylation, the attachment of a geranylgeraniol group; and glypiation, glycosylphosphatidylinositol (GPI) anchor formation via an amide bond.

The modification with a cofactor is preferably selected from lipoylation, attachment of a lipoate (C8) functional group; flavination, attachment of a flavin moiety (e.g. flavin mononucleotide (FMN) or flavin adenine dinucleotide (FAD)); attachment of heme C, for instance via a thioether bond with cysteine; phosphopantetheinylation, the attachment of a 4′-phosphopantetheinyl group; and retinylidene Schiff base formation. The addition of a chemical group is preferably selected from acylation, e.g. O-acylation (esters), N-acylation (amides) or S-acylation (thioesters); acetylation, the attachment of an acetyl group for instance to the N-terminus or to lysine; formylation; alkylation, the addition of an alkyl group, such as methyl or ethyl; methylation, the addition of a methyl group for instance to lysine or arginine; amidation; butyrylation; gamma-carboxylation; glycosylation, the enzymatic attachment of a glycosyl group for instance to arginine, asparagine, cysteine, hydroxy lysine, serine, threonine, tyrosine or tryptophan; polysialylation, the attachment of polysialic acid; malonylation; hydroxylation; iodination; bromination; citrullination; nucleotide addition, the attachment of any nucleotide such as any of those discussed above, ADP ribosylation; oxidation; phosphorylation, the attachment of a phosphate group for instance to serine, threonine or tyrosine (O-linked) or histidine (N-linked); adenylylation, the attachment of an adenylyl moiety for instance to tyrosine (O-linked) or to histidine or lysine (N-linked); propionylation; pyroglutamate formation; S-glutathionylation; Sumoylation; S-nitrosylation; succinylation, the attachment of a succinyl group for instance to lysine; selenoylation, the incorporation of selenium; and ubiquitinoylation, the addition of ubiquitin subunits (N-linked).

The addition of a chemical group may concern any non-natural chemical modification of one or more cysteines, lysines, tyrosines, arginines or any other (natural or not) residue within the peptide, polypeptide or protein.

A “nanopore” is any structure comprising and/or defining a pore or opening having a diameter of less than 1 micron, typically between 1 and 20 nm in diameter or width (for example, internal diameter or internal width), for example between 2 and 5 nm in diameter (extremity values of 2 nm and 5 nm included). As a way of example for the sake of providing reference dimensions, single stranded DNA can pass through a 2 nm nanopore, whereas double stranded DNA can pass through a 4 nm nanopore. Having a very small nanopore, e.g., 2-5 nm, allows a molecule or biomolecule ((bio)molecule) such as DNA to pass through, but not larger molecular entities such as proteinaceous complexes or enzymes, thereby allowing for controlled passage of charged polymers or (bio)molecules in general.

The nanopore may, for example, extend fully or partially through the structure or material in which the nanopore is defined or comprised.

Different types of nanopores are known. For example, biological nanopores are formed by assembly of (a) pore-forming protein(s) in a membrane such as a lipid bilayer. For example, α-hemolysin and similar protein pores (MspA, aerolysin etc) are found naturally in cell membranes, where they act as channels for ions or molecules to be transported in and out of cells, and such proteins can be repurposed as nanochannels.

Solid-state nanopores are formed in synthetic materials such as silicon nitride, glass or graphene, by e.g. configuring holes or bores in the synthetic membrane, using for instance feedback controlled low energy ion beam sculpting (IBS) or high energy electron beam illumination. Hybrid nanopores can be made by embedding a pore-forming protein in synthetic materials. The present invention concerns methods and systems using, for example, solid state nanopores or hybrid nanopores, or preferably nanopores obtained in glass capillaries.

Where there is a means for applying an electrical potential at either end or either side of a nanopore via e.g. electrodes, a current flow across the nanopore may be established through the nanopore, possibly through an electrolyte media. Electrodes may be made of any conductive material, for example silver, gold, platinum, copper, titanium dioxide, for example silver coated with silver chloride. The flow of materials across a nanopore may also be regulated by electrodes; for example, as (bio)molecules are electrically charged, or may be electrically charged depending on some factors such as the pH of the medium they are in (e.g., DNA and RNA are negatively charged in many buffer media), they will be drawn to a positively charged electrode upon application of an electrical voltage across the nanopore. In the event a polymer passes through the nanopore, the change in electric potential, capacitance or current across the nanopore caused by the partial blockage of said nanopore can be detected and used to identify e.g. the sequence of monomers in the polymer, wherein different monomers can be distinguished by their different sizes and/or electrostatic potentials. When reference is made to a “(bio)molecule” or “(bio) polymer” and the like, said (bio)molecule and/or (bio) polymer is, for example, electrically charged, that is, it has a net positive or negative charge in the medium it is comprised in, said net positive or negative charge being such that said (bio)molecule and/or (bio) polymer can be flowed or drawn into, and/or retained in a nanopore structure.

The terms “membrane”, “film” or “thin film” can be used interchangeably and relate to the thin form factor of an element of the device of the invention. Generally speaking, a “membrane”, “film” or “thin film” as used herein relate to a layer of a material having a thickness much smaller than the other dimensions, e.g. at least one fifth compared to the other dimensions. Typically, a film is a solid layer having a first surface and an opposed second surface, with any suitable shape, and a thickness generally in the order of nanometers or micrometers, depending on the needs and circumstances, e.g. the manufacturing steps used to produce it. In some embodiments, films according to the invention have a thickness comprised between 0.1 nm to 500 μm, such as between 0.3 and 10 nm, between 1 and 50 nm, between 20 and 100 nm, between 200 and 500 nm, between 50 nm and 1 μm, between 1 and 50 μm, between 50 μm and 150 μm, between 100 μm and 500 μm or between 200 μm and 500 μm (extremity values of the above ranges included).

In embodiments of the invention, a membrane or thin film can be made of a silicon material, for example silicon dioxide or silicon nitride. Silicon nitride (e.g., Si₃N₄) is especially desirable for this purpose because it is chemically relatively inert and provides an effective barrier against diffusion of water and ions even when only a few nm thick. Silicon dioxide is also useful, because it is a good surface to chemically modify. Alternatively, in certain embodiments, a membrane or thin film may be made in whole or in part out of materials which can form sheets as thin as a single molecule (sometimes referred to as “single layered” membrane, “monolayer” membrane or “2D” and “two dimensional” sheet or membrane), for example and without limitation: graphene; GaS; GaSe; GaTe; MX₂ type of dichalcogenides where M=Mo, Nb, Ni, Sn, Ti, Ta, Pt, V, W, or Hf and X═S, Se, or Te; M₂X₃ type of trichalcogenides where M=As, Bi, or Sb and X═S, Se, or Te; MPX₃ where X═S or Se; MAX3 where A=Si or Ge and X═S, Se, or Te; and alloy sheets like MₓM′₁₋ₓS₂, as well as combinations of any of the foregoing. Accordingly, suitable materials include molybdenum disulfide (MoS₂), molybdenum diselenide (MoSe₂), molybdenum ditelluride (MoTe₂), tungsten disulfide (WS₂), tungsten diselenide (WSe₂), tungsten ditelluride (WTe₂), chromium disulfide (CrS₂), chromium diselenide (CrSe₂), chromium ditelluride (CrTe₂), gallium arsenide, germanium, boron nitride (hBN) and gallium indium phosphide. Solid-state nanopores are, for example, included or defined in the membrane or thin film to form a probe structure.

In other embodiments of the invention, a nanopore can be located at one end of a glass capillary, or a capillary or capillaries substantially made of other kinds of inert materials. Advantageously, the form factor of a capillary design generally facilitates the targeting and capturing of target (polymeric) (bio)molecules through the nanopore by an electrophoretic force, as will be detailed later on in the examples part of the present disclosure.

A “two-dimensional” or “2D” layer, sheet, polymer, film, membrane and the like is a sheet-like, macromolecule of elements or crystal having a thickness in the order of a single molecule (monomolecular) layer, i.e. of a few nanometres or less, and are therefore not retrievable in nature as free-standing structures. The most known example of a two-dimensional crystal is graphene, an individual, atomically thin layer or sheet of graphite. However, in a broader sense, a 2D structure may comprise more than one monolayer, such as two or three stacked monomolecular layers, and still be considered as two-dimensional in nature. Two-dimensional materials, sometimes also referred to as layered materials, may comprise laterally connected repeat units (monomers) or may be composed of a single or few atomic elements. These materials have found use in applications such as photovoltaics, semiconductors, electrodes and water purification, to cite a few. Layered combinations of different 2D materials are generally called van der Waals heterostructures, and can be used as a structure that includes one or more nanopores or solid-state nanopores in the frame of the present invention.

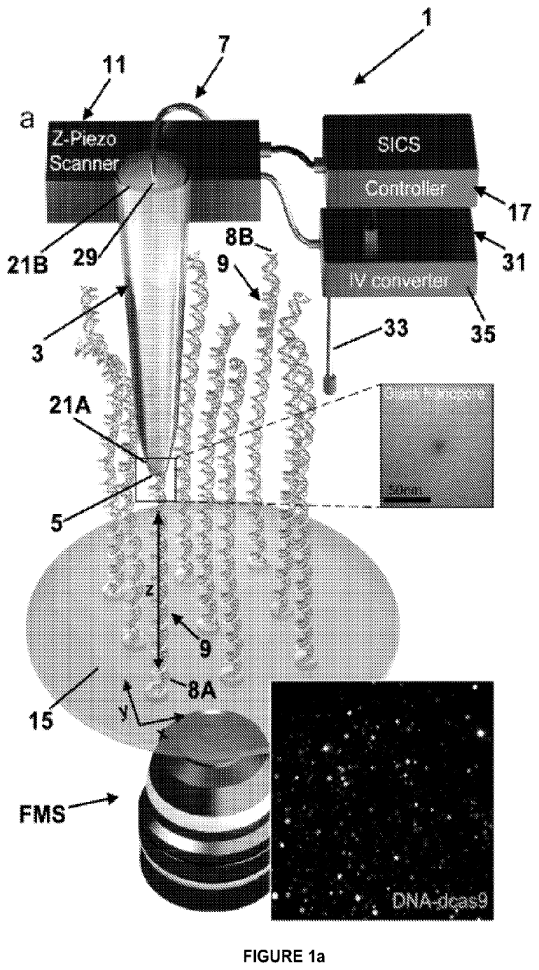

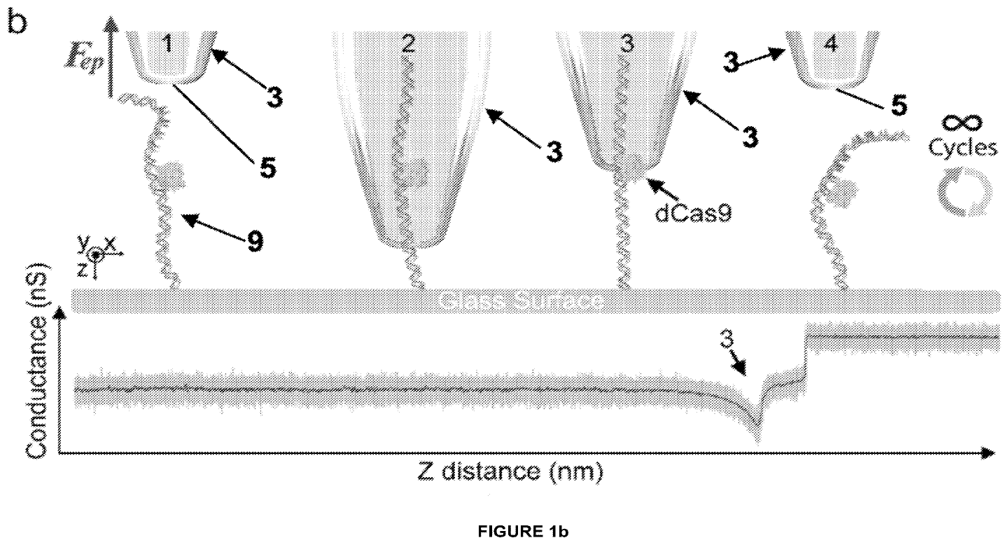

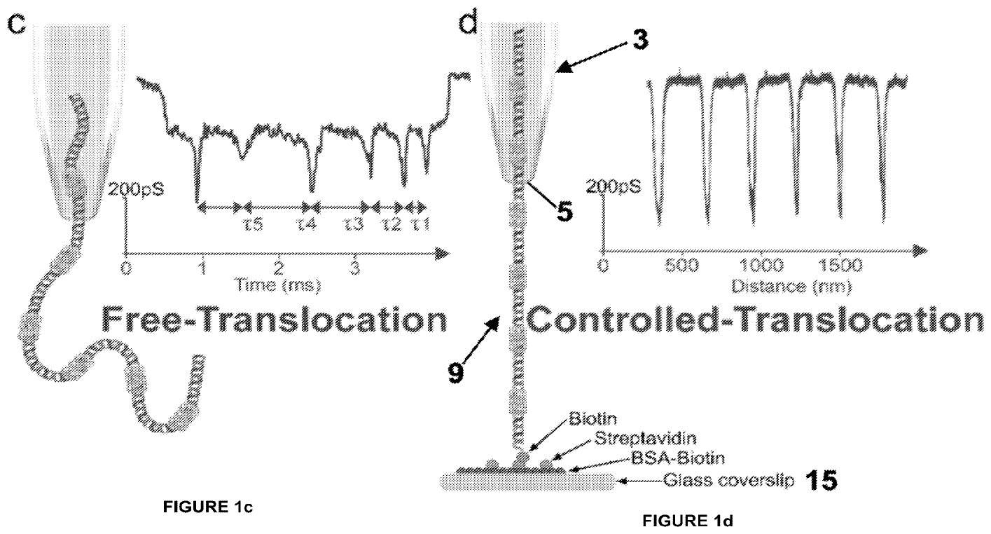

In the following description, an orthogonal reference frame XYZ is defined with three axes perpendicular to each other (see, for example, FIG. 1a), namely:
an X axis, defining a longitudinal, (substantially) horizontal direction,
a Y axis, defining a transverse, (substantially) horizontal direction, with which the X axis defines a (substantially) horizontal XY plane,
a Z axis, defining a transverse, (substantially) vertical direction, perpendicular to the XY plane.

FIG. 1a is a schematic illustration of an exemplary nanopore-based scanning system 1 or scanning ion conductance spectroscopy (SICS) system 1 according to the present disclosure.

The system 1, may for example optionally be combined with or further include a fluorescence microscopy system FMS.

The nanopore-based scanning system 1 may include, for example, at least one probe structure 3 comprising at least one nanopore 5, suction or taking-in means 7 configured to draw, for example single or multiple times, an end 8B of at least one (bio)molecule 9 inside the at least one nanopore 5 and inside the at least one probe structure 3.

The system 1 may further include displacement means 11 or a displacer 11 configured to mechanically displace the at least one probe structure 3 and the at least one nanopore 5 relative to the (bio)molecule 9 while the (bio)molecule 9 is located inside the at least one nanopore 5 and inside the at least one probe structure 3.

The nanopore-based scanning system 1 may, for example, comprise, at least one support 15 configured to physically tether on a surface thereof the one or more (bio)molecules 9. In this context, the support 15 may be for instance chemically functionalized to physically tether on a surface thereof the one or more (bio)molecules 9.

The displacement means 11 is, for example, configured to mechanically displace the probe structure 3 and the nanopore 5 relative to the (bio)molecule 9 along a direction following a direction of extension of the (bio)molecule 9 while the (bio)molecule 9 is located inside the nanopore 5 and inside the probe structure 3. The displacement of the probe structure 3 and the nanopore 5 is carried out with respect to the (bio)molecule 9 that is attached or held on the support 15, with the support 15 being immobile or held stationary with respect to the probe structure 3 and the nanopore 5.

The displacement means 11 is, for example, configured to mechanically displace the probe structure 3 and the nanopore 5 relative to the (bio)molecule 9 attached to the support 15 held stationary.

The displacement means 11 or displacer 11 may, for example, include a mobile element and an attachment or fastener configured to attach or fix or hold the probe structure 3 to the mobile element that is mobile to displace the probe structure 3 and the nanopore 5 relative to or with respect to the support 15 and/or the (bio)molecule 9 to permit controlled and/or deterministic displacement of the probe structure 3 and the nanopore 5 relative to the support 15 and/or the (bio)molecule 9.

Alternatively, or additionally, the displacement means 11 is, for example, configured to hold the support 15 and to simultaneously mechanically displace the support 15 (holding the one or more (bio)molecules 9) relative to the probe structure 3 and the nanopore 5, to mechanically displace the support 15 along the direction following a direction of extension of the (bio)molecule 9 while the (bio)molecule 9 is located inside the nanopore 5 with displacement being carried out while the support 15 is simultaneously being held. The probe structure 3 and the nanopore 5 are, for example, held immobile or held stationary with respect to the support 15.

The displacement means 11 is, for example, configured to mechanically displace the support 15 holding the (bio)molecule or the (bio)molecules 9 relative to the nanopore 5 and probe structure 3 held stationary.

The attachment or fastener is configured to attach or fix or hold the support 15 to the mobile element that is mobile to displace the support 15 (and/or the (bio)molecule 9) relative to the probe structure 3 and the nanopore 5 to permit controlled and/or deterministic displacement of the support 15 (and/or the (bio)molecule 9) relative to the probe structure 3 and the nanopore 5.

The attachment or fastener may, for example, be a chemical attachment or fastener implemented by an adhesive, or a surface contact based attachment or fastener implemented by direct or indirect contact with a surface of a further object, for example, the mobile element. Non-limiting embodiments of the attachment or fastener may, for example, comprise a form-fit holder, a press-fit holder, a threadedly engaging enclosure or buttress, or alternatively an adhesive.

The nanopore-based scanning system 1 may comprise, for example, the at least one support 15. The support 15 is, for example, configured to physically tether on a surface thereof the at least one (bio)molecule 9. In this context, the support 15 may be for instance chemically functionalized to physically tether on a surface thereof the at least one (bio)molecule 9.

The displacement means 11 is configured to mechanically displace the probe structure 3 and the nanopore 5 in a forward and reverse direction (or, for example, up and down) along a direction of extension of the at least one (bio)molecule 9 while the at least one (bio)molecule 9 is located inside the nanopore 5 and inside the probe structure 3. Preferably, the displacement means 11 is configured to mechanically displace the probe structure 3 and the nanopore 5 relative to the support 15 configured to physically tether on a surface thereof the at least one (bio)molecule 9, or vice-versa.

Still preferably, the displacement means 11 is configured to mechanically displace the probe structure 3 and the nanopore 5 relative to the support 15 configured to physically tether on a surface thereof the at least one (bio)molecule 9, or vice-versa, in a forward and reverse direction along a direction of extension of the at least one (bio)molecule 9 while the at least one (bio)molecule 9 is located inside the nanopore 5 and inside the probe structure 3, and wherein the at least one (bio)molecule 9 is physically tethered on a surface of the support 15.

The (bio)molecule 9 or each (bio)molecule 9 includes, for example, a first end 8A attached to the support 15 and a second non-attached end 8B that is free to move while the (bio)molecule 9 is anchored to the support 15.

The displacement means 11 is configured to mechanically displace the probe structure 3 and the nanopore 5 in a forward and reverse direction along a direction of extension of the (bio)molecule 9 and towards and away from the support 15 while the (bio)molecule 9 is located inside the nanopore 5 and inside the probe structure 3, and when the (bio)molecule 9 is attached to the support 15.

The displacement means 11 is configured to mechanically displace the probe structure 3 and the nanopore 5 towards a surface of the support 15 and the attached end 8A of the (bio)molecule 9 while the (bio)molecule 9 is located inside the nanopore 5 and inside the probe structure 3. The displacement means 11 is configured to mechanically displace the probe structure 3 and the nanopore 5 away from the surface of the support 15 towards the non-attached end 8B of the (bio)molecule 9 and to pass the non-attached end 8B out of the nanopore 5 and the probe structure 3.

The displacement means 11 is, for example, configured to mechanically displace the probe structure 3 and/or the support 15 in one or a plurality of directions X, Y, Z, for example, in the above-mentioned orthogonal reference frame XYZ defined with three axes perpendicular to each other.

The displacement means 11 is, for example, configured to deterministically control a speed and direction of displacement of the probe structure 3 and/or the support 15 in one or a plurality of directions X, Y, Z, for example, in the above-mentioned orthogonal reference frame XYZ defined with three axes perpendicular to each other.

The displacement means 11 is, for example, configured to displace the probe structure 3 (and the nanopore 5) and/or the support 15 at a (substantially) constant or defined velocity of displacement, for example in the vertical Z-direction, to displace the (bio)molecule 9 at a (substantially) constant velocity of displacement through and relative to the nanopore 5.

The displacement means 11 or the displacer 11 may, for example, comprise at least one or a plurality of piezoelectric positioners or stages (such as piezo crystal nano-positioner(s)) configured to permit displacement in or along the one or the plurality of axes or directions X, Y, Z. While the exemplary embodiment illustrated in FIG. 1a shows a piezoelectric positioner configured to displace the probe structure 3 in the Z-direction, additional piezoelectric positioners may also be included to additionally displace the probe structure 3 in the X-direction and the Y-direction or alternatively displace the support 15 in the X-direction and the Y-direction. Relative displacement may, for example, be alternatively assured using piezoelectric positioners configured to displace the support 15 in X, Y and Z directions.

The piezoelectric positioner includes the above-mentioned mobile element and may, for example, also include the attachment or fastener configured to attach or fix the probe structure 3 or the support 15.

The system 1 also includes a system controller 17 configured to control displacements of the probe structure 3 and/or the support 15 holding the (bio)molecules 9. The system controller 17 includes, for example, one or a plurality of piezoelectric positioner controllers, for example, one for each directional control.

The probe structure 3 may comprise or consist of, for example, at least one glass (or inert material) capillary probe, or glass (or inert material) capillary pipette. The probe structure 3 may comprise or consist of, for example, a nanopipette or micropipette. The probe structure or capillary probe 3 may, for example, comprise an elongated hollow body or tube 19 to receive the (bio)molecules 9 therein, the elongated hollow body 19 including a first open extremity 21A comprising or defining the nanopore 5 and a second (open) extremity 21B located opposite the first open extremity 21A. The elongated hollow body 19 extends, for example, to have a length permitting the (bio)molecule 9 to be received fully inside the elongated hollow body 19. The elongated hollow body 19 may, for example, have a conical shape as shown in the illustrated example of FIG. 1a. However, other shapes are also possible as mentioned previously. Characteristics of the nanopore 5 have also been described previously herein.

The probe structure 3 may, alternatively, comprise a biological nanopore, or a solid-state nanopore, as described previously herein.

The suction or taking-in means 7 means is, for example, configured to draw a free end 8B of a tethered or attached (bio)molecule 9 into and/or through the nanopore 5 to capture the tethered (bio)molecule 9 inside the probe structure 3.

The suction or taking-in means 7 (or aspirator configured to draw or suck an end 8B of the (bio)molecule 9 inside the nanopore 5 and inside the probe structure 3) comprises, for example, an electrophoretic force generator configured to generate an electrophoretic force to draw or suck an end 8B of the (bio)molecule 9 inside the nanopore 5 and inside the probe structure 3.

The suction or taking-in means 7 includes, for example, at least one electrode 29 included inside the probe structure 3 and/or attached to or on the probe structure 3 to which a bias voltage is applied to (or is to be applied to), and acts as an electrophoretic force generator that generates an electrophoretic force that draws a free end 8B of the (bio)molecule 9 through the nanopore 5 and inside the probe structure 3.

The suction or taking-in means 7 may further include at least one power supply 31 (for example, a voltage source) configured or operatively connected to the at least one electrode 29 to apply a voltage thereto to generate an electrophoretic force.

The power supply 31 may also be configured or operatively connected to the at least one electrode 29 to apply a voltage difference to the probe structure 3. The voltage difference is, for example, applied between the probe structure 3 and the support 15.

The nanopore-based scanning system 1 may further include at least one electrode 29, or a first electrode 29 and a second or reference electrode 33 arranged to provide an ionic-current measurement during displacement of the probe structure 3 relative to the (bio)molecule 9, or during the relative displacement between the probe structure 3 and the support 15 containing the (bio)molecule 9.

The displacement means 11 is configured to operate a relative displacement of the probe structure 3 compared to the support 15 configured to be functionalized to attach the at least one (bio)molecule thereto, the displacement means 11 being configured to operate a mechanical displacement of the probe structure 3 and/or the support 15 in a least one or a plurality of directions (X, Y, Z).

The second or reference electrode 33 may, for example, be located inside an electrolyte in which the (bio)molecules 9 are located (the electrolyte is not shown in FIG. 1a to allow the (bio)molecules 9 to be visible). The support 15 may, for example, be a base of a container holding the electrolyte, or alternatively be a support placed inside a container holding the electrolyte. The first electrode 29 of the probe structure 3 is, for example, also arranged or located on the probe structure 3 so as to be in contact with the electrolyte when being displaced along the (bio)molecules 9.

The first and second electrodes 29, 33 are, for example, connected to the power supply 31 so that a voltage difference is applied to the first and second electrodes 29, 33 permitting an ionic-current to flow between the electrodes 29, 33 via the electrolyte, and permitting to measure a ionic-current variation or conductance value as the nanopore 5 is displaced along the (bio)molecule 9, the displacement of the (bio)molecule 9 through the nanopore 5 modifying the ionic-current flow. Additional electrodes may also be present to assure measurement of the current.

The displacement means 11 may, for example, be configured to provide a measure or value representing a distance displacement of the probe 3 and nanopore 5, for example, a distance from the support 15 and/or along the (bio)molecule 9. For example, the piezoelectric positioner controller is for example configured to measure or provide a value representing this distance displacement.

The nanopore based scanning system 1 is, for example, configured to detect (determine) and/or control a distance between the probe structure 3 and the substrate/support 15 based on the (ionic) current flowing through the nanopore 5 that is measured by the system 1. This permits to know where the surface of the support 15 is located with respect to the nanopore 5. For example, a change or drop in current is observed when the nanopore is located close to the surface of the substrate 15. This can, for example, also be done when the (bio)molecule 9 is located inside the probe structure 3.

This permits, for example, to determine a starting location or position from which the probe structure 3 is displaced along the (bio)molecule 9 to obtain a characteristic signal or a characterising measurement or signature of the (bio)molecule 9.

The nanopore-based scanning system 1 is, for example, configured to carry out a controlled translocation by drawing a (bio)molecule 9 inside the nanopore 5 and inside the probe structure 3 and mechanically displacing the probe structure 3 and the nanopore 5 forward and/or backwards along a direction of extension of the (bio)molecule 9 while the (bio)molecule 9 is located inside the nanopore 5 and inside the probe structure 9.

The nanopore-based scanning system 1 may include the (bio)molecule 9 or the (bio)molecules 9.

The (bio)molecule 9 may comprise or consist of a polymeric molecule.

The (bio)molecule 9 may comprise or consist of oligonucleotides, or polynucleotides (nucleic acids), or polypeptides, or oligopeptides, or hybrid nucleic acid/peptide constructs, or fatty acids, or glycans, or any combination thereof.

The nanopore-based scanning system 1 may comprise at least one or a plurality of arrays of probes structures 3, and/or at least one or a plurality of arrays of (bio)molecules 9 located on the at least one support 15.

The support 15 may comprise or consist of glass or transparent/translucent material permitting fluorescence measurements of the (bio)molecule or (bio)molecule 9.

The nanopore-based scanning system 1 may further include a fluorescence microscope or system FMS configured to perform fluorescence measurements of the (bio)molecule 9 or (bio)molecules 9.

According to an another embodiment, the displacement means 11 may be configured to mechanically displace the probe structure 3 and the nanopore 5 towards or away from the (bio)molecule 9 along a direction following a direction of extension of the (bio)molecule 9 while the (bio)molecule 9 is located inside the nanopore 5 and inside the probe structure 3, and also configured to simultaneously mechanically displace the support 15 holding the (bio)molecule 9 towards or away from the nanopore 5 and the probe structure 3 along the direction following a direction of extension of the (bio)molecule 9 while holding support 15 and while the (bio)molecule 9 is located inside the nanopore 5.

Another aspect of the present disclosure concerns a point-of-care device including the nanopore-based scanning system 1.

A further aspect of the present disclosure concerns a nanopore-based scanning method including providing the nanopore-based scanning system 1; and drawing the (bio)molecule 9 or each (bio)molecule 9 inside the nanopore 5 and inside the probe structure 3 and then mechanically displacing the probe structure 5 and the nanopore 3 forward and/or backwards along a direction of extension of the (bio)molecule 9 while the (bio)molecule 9 is located inside the nanopore 5 and inside the probe structure 3, the (bio)molecule 9 being, for example, attached on the support 15 movable relative to the at least one probe structure 3.

Such displacement assures a controlled and/or deterministic displacement of the probe structure 3 and the nanopore 5 relative to the support 15 and/or the (bio)molecule 9.

Yet another aspect of the present disclosure concerns a nanopore-based scanning method including providing at least one support 15 comprising at least one or a plurality of (bio)molecules 9 attached thereto; carrying out a controlled translocation or a controlled and/or deterministic displacement of the probe structure 3 and the nanopore 5 relative to the support 15 and/or the (bio)molecule 9 by drawing or sucking an end 8B of the at least one (bio)molecule 9 inside the nanopore 5 of the probe structure 3 and inside the probe structure 3; and mechanically displacing the probe structure 3 and the nanopore 5 relative to the support 15, or vice versa, and along a direction of extension of the (bio)molecule 9 while the (bio)molecule 9 is located inside the nanopore 5 to displace the (bio)molecule 9 through the nanopore 5.

The probe structure 3 and the nanopore 5 may be mechanically displaced relative to the support 15, for example, held stationary, and along a direction of extension of the (bio)molecule 9 while the (bio)molecule 9 is located inside the nanopore 5 to displace the (bio)molecule 9 through the nanopore 5.

Additionally or alternatively, the support 15 holding the (bio)molecule or (bio)molecules 9 may be mechanically displaced relative to the nanopore 5 and probe structure 3 held stationary, and displaced along a direction following a direction of extension of the (bio)molecule (9) while holding the support 15 during displacement and while the (bio)molecule 9 is located inside the nanopore 5.

According to an another embodiment, the probe structure 3 and the nanopore 5 may be mechanically displaced towards or away from the (bio)molecule 9 along a direction following a direction of extension of the (bio)molecule 9 while the (bio)molecule 9 is located inside the nanopore 5 and inside the probe structure 3, and the support 15 holding the (bio)molecule 9 may also or simultaneously be mechanically displaced towards or away from the nanopore 5 and the probe structure 3 along the direction following a direction of extension of the (bio)molecule 9 while holding support 15 and while the (bio)molecule 9 is located inside the nanopore 5.

A voltage difference can be applied between (i) at least one electrode 29 of the probe structure 3 and (ii) the at least one support 15, and an ionic-current variation or conductance can be determined/measured as the nanopore 5 is mechanically displaced along the direction of extension of the (bio)molecule 9 to detect or locate (bio)molecule features.

This permits, for example, to obtain a characteristic signal or a characterising measurement or signature of the (bio)molecule 9. This may be used, for example, to identify or determine constituent elements or the make-up of the (bio)molecule 9. This may be done, for example, through comparison with known signatures measured for known the (bio)molecule 9, for example, recorded in a digital library.

The probe structure 3 and the nanopore 5 can be mechanically displaced forward and/or backwards relative to the support 15, or vice versa, and along a direction of extension of the (bio)molecule 9 while the (bio)molecule 9 is located inside the probe structure 3.

The probe structure 3 and the nanopore 5 can, for example, be displaced and the support 15 is held stationary, or the support 15 can be displaced and the probe structure 3 and the nanopore 5 is held stationary.

An electrophoretic force can be generated by an applied voltage bias to draw or suck an end 8B of the (bio)molecule 9 inside the nanopore 5 and inside the probe structure 3.

The controlled translocation can be carried out at a constant or defined velocity of displacement of the nanopore 5.

The controlled translocation can be carried out multiple times on the same (bio)molecule 9 to obtain a plurality of ionic-current variation measurements or conductance measurements as the nanopore 5 is mechanically displaced along the direction of extension of the (bio)molecule 9, and an average measurement can be determined.

Bi-directional controlled translocations can, for example, be carried out to obtain bi-directional ionic-current variation or conductance measurements.

Controlled translocations can be carried out on a plurality of (bio)molecules 9 attached across the support 15.

The at least one support 15 can be functionalized and include at least one or a plurality of (bio)molecules 9 tethered thereto.

Fluorescence measurements can also be carried out of the at least one or the plurality of (bio)molecules 9.

The probe structure 3 may comprise or consist of at least one glass capillary probe or pipette, or at least one probe including a biological nanopore, or at least one probe including a solid-state nanopore.

The probe structure 3 may comprise or consist of at least one glass nanopipette or micropipette.

The (bio)molecule 9 may comprise or consist of a polymeric molecule.

The (bio)molecule 9 may comprise or consist of oligonucleotides, or polynucleotides (nucleic acids), or polypeptides, or oligopeptides, or hybrid nucleic acid/peptide constructs, or fatty acids, or glycans, or any combination thereof.

At least one or a plurality of arrays of probes structures 3 may be provided, and/or at least one or a plurality of arrays of (bio)molecules 9 located on the at least one support 15 may be provided.

The support 15 may comprise or consist of glass or transparent/translucent material permitting fluorescence measurements of the at least one (bio)molecule 9.

The relative distance between the probe structure 3 and the support 15 can be detected and/or controlled based on the current or ionic-current through the nanopore 5. For example, the current or ionic-current is reduced as the probe structure and nanopore 5 approaches closer to the support 15.

The system and method according to the invention are also suitable for detection and analysis of post-translational modifications (PTMs) of oligo-, polypeptides, proteins and hybrid nucleic acid/peptide constructs. The detection can be done also on or at the single molecule level and change can be induced also in-situ on the same molecule.

A presence, or an absence, or a number or a position of one or more post-translational modifications PTMs can be determined in a peptide, polypeptide, protein or hybrid nucleic acid/peptide construct 9 by passing the peptide, polypeptide, protein or hybrid nucleic acid/peptide construct through the nanopore 5 and measuring one or more ionic-current measurements as previously described. A Post-translational modification PTM may be, for example, a modification with a hydrophobic group, or a modification with a cofactor, or an addition of a chemical group, or glycation, or biotinylation or pegylation, or a non-natural chemical modification for biotechnological or biomedical purposes.

In order to implement the methods of the present invention, the system 1 may comprise an operatively coupled computing device configured to control the operation of the system 1, said computing device comprising a memory and a processing unit encoding instructions that, when executed, cause the processing unit to control means to carry out the method of the present disclosure; to control, for example, at least one of means to provide a voltage, suction means, means for recording and analyzing an electrical current, and means for moving a nanopore-based device or probe structure 3 comprising the nanopore 5 relative to target polymeric molecules 9. The computing device may include one or more processing units and computer readable media. Computer readable media includes physical memory such as volatile memory (such as RAM), non-volatile memory (such as ROM, flash memory, etc.) or some combination thereof. Additionally, the computing device can include mass storage (removable and/or non-removable) such as a magnetic or optical disks or tape. An operating system and one or more application programs can be stored on the mass storage device. The computing device can further include input devices (such as a keyboard and mouse) and output devices (such as a monitor), if needed.

More specific details of a non-limiting exemplary embodiment of the invention of the present disclosure is now described. FIG. 1a is a schematic of the non-limiting exemplary embodiment of the nanopore-based scanning system 1 or SICS system 1 according to the present disclosure.

The system 1 includes a probe structure 3 comprising a nanopore 5 which can be displaced in a Z-direction using for example displacement means 11 such as a piezo positioner. A support 15 including a plurality of (bio)molecules 9 can, for example, be displaced by one or more further piezo positioners in the X-direction and the Y-direction. The support 15 may be functionalized to attach the (bio)molecules, for example, using the streptavidin/biotin system. Suction means 7 configured to draw an end 8B of a (bio)molecule 9 inside the nanopore 5 and inside the one probe structure 3 is also present.

The suction means 7 comprises for example a first electrode 29 included inside and/or on the probe structure 3 to which a bias voltage is applied to and acts as an electrophoretic force generator that generates an electrophoretic force that draws a free end 8B of the (bio)molecule 9 through the nanopore 5 and inside the probe structure 3.

A second or reference electrode 33 is also included and is for example located inside an electrolyte in which the (bio)molecules 9 are located. The first and second electrodes 29, 33 are also used to measure a ionic-current variation or conductance value as the nanopore 5 is displaced along the (bio)molecule 9. The measurement of the current could also include other electrodes such as for example reference electrodes. The system 1 may include a current to voltage converter 35 and a power/voltage source 31. The system also includes one or more controllers 17 configured to control displacements of the probe structure 3 and the support 15 holding the (bio)molecules 9.

In an implemented, non-limiting embodiment, controlled-translocations with the SICS 1 used an electrophoretic force (F_{elec}) generated by a bias applied (ΔV) to capture DNA molecules tethered on a glass surface 15, characterized by a negative bare line charge density (λ_{bare}=−0.96 nC from 2 electrons per base pair), with F_{elec}∂λ_{bare}ΔV.

In a SICS experiment, the electrophoretic force captured the DNA when the probe 3 approaches the free-end 8B of a glass-tethered molecule 9 (FIG. 1b-1).

The distance between the nanopore 5 and the surface can be determined through the measurement of the current as a function of distance, in a similar way as common in SICM.

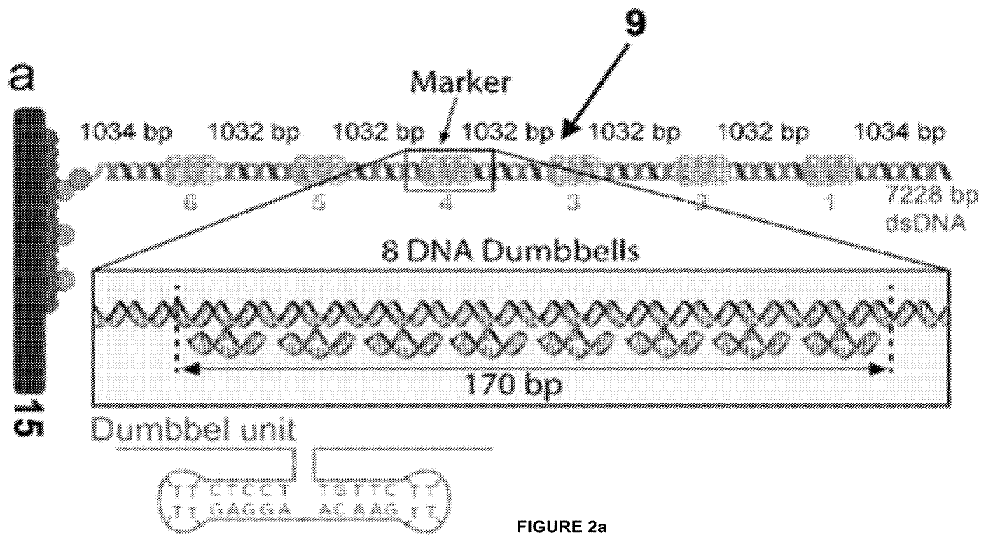

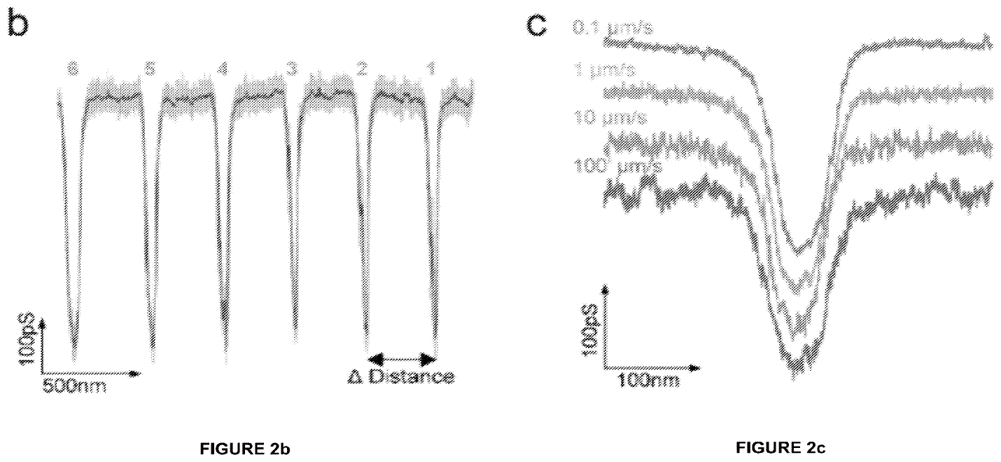

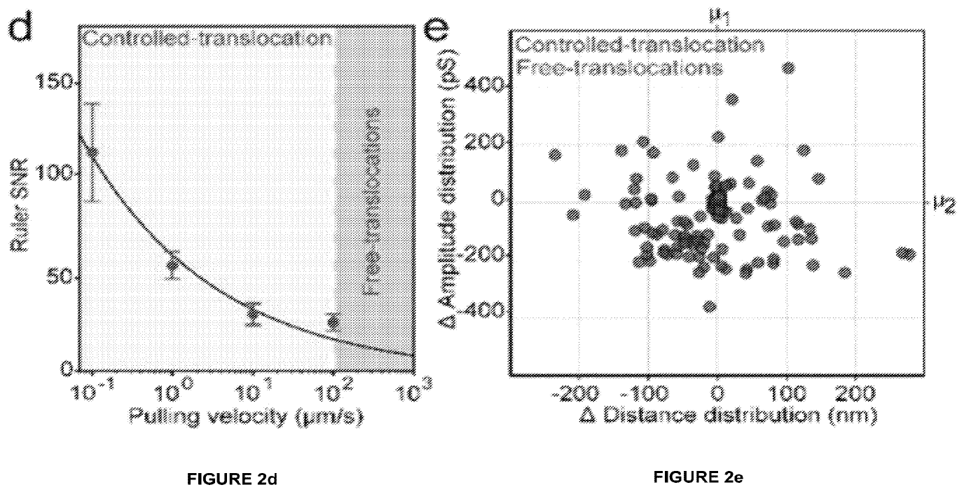

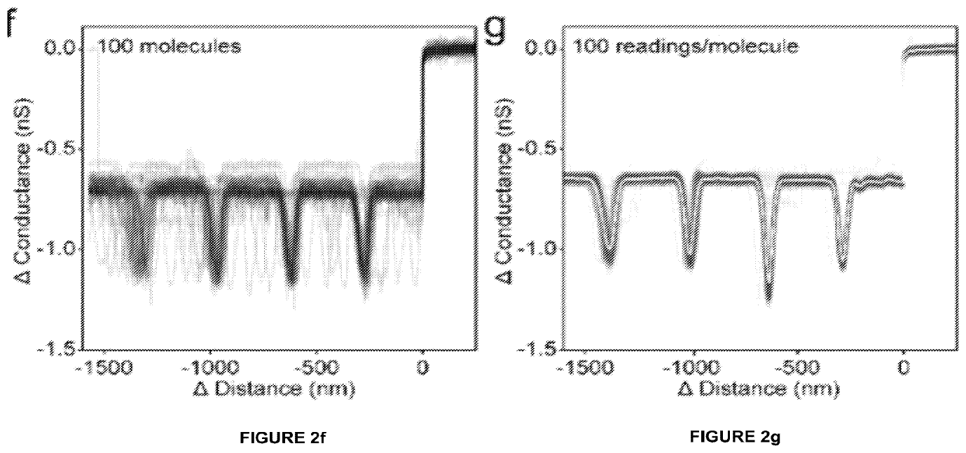

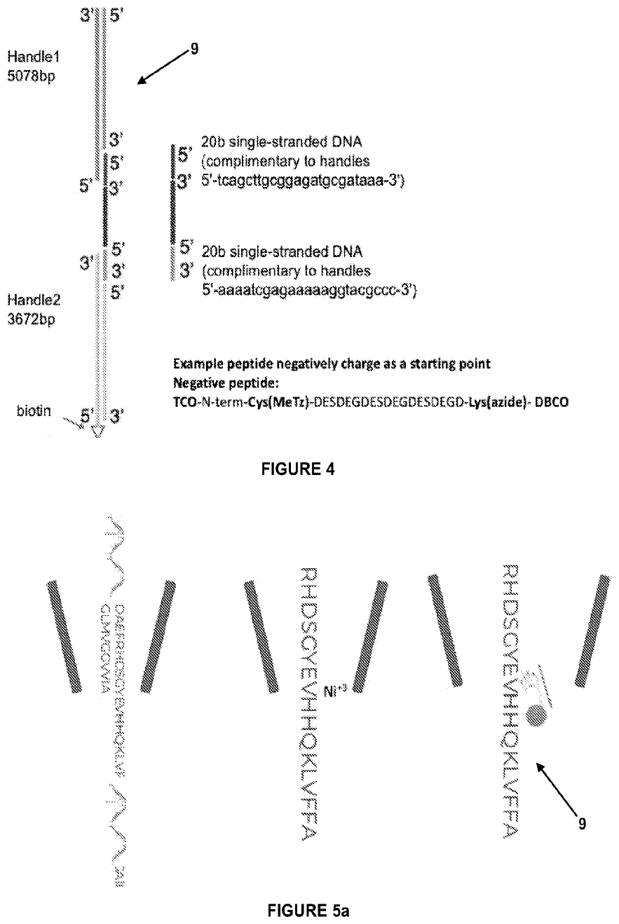

Once the molecule 9 is inside the pore 5, the SICS probe 3 performs a controlled-translocation via a piezo crystal nano-positioner that moves the pore 3 at a desired rate or velocity (for example constant velocity) with picometer precision from the glass surface (FIG. 1b-2). The data generated corresponds to a conductance-distance curve that reveals features along the dsDNA length (FIG. 1b-3). The approach of the present disclosure advantageously allows translocating thousands of molecules 9 tethered on a surface and/or thousands readings of the same molecule 9 (FIG. 1b-4).

To benchmark the performance of the system 1, the inventors compared the SNR of free-translocations (FIG. 1c) with controlled-translocations (FIG. 1d). The inventors performed experiments on a custom-designed DNA ruler, composed of a 7′228 base pairs DNA strand (2′458 nm) and 6 markers of DNA dumbbell hairpins which are positioned at 1′032 base pairs intervals along the DNA contour (FIG. 2a). FIG. 2b shows the conductance-distance curve generated by SICS, revealing 6 markers.

Controlled-translocation with the SICS 1 allows decreasing the speed by more than 4 orders of magnitude compared with typical free-translocations, leading to an increased SNR (FIG. 2c). At the lowest velocity of 0.1 μm/s the SNR measured was 141.8+/−8 with an 8 nm glass nanopore radius (FIG. 2d). This corresponds to an improvement of SNR on a single reading by 10 fold compared with a free-translocation (>100 μm/s velocity).

Controlling the translocation dynamics with the SICS 1, allowed the method to detect and locate features along the DNA molecule with high precision. FIG. 2e shows a location precision above 99% with controlled-translocations, compared with 10% in free-translocations with glass nanopores 3 of the same pore size. The SICS 1 can map out diverse molecules 9 tethered on the surface or/and independently selected molecules 9. FIG. 2f shows the overlay of 100 distinct molecules of the designed DNA ruler and FIG. 2g shows 100 readings of the same selected molecule 9. It was observed minimal differences in the dumbbell structure, preserving the spatial characteristics of individual DNA dumbbells at the single-molecule level.

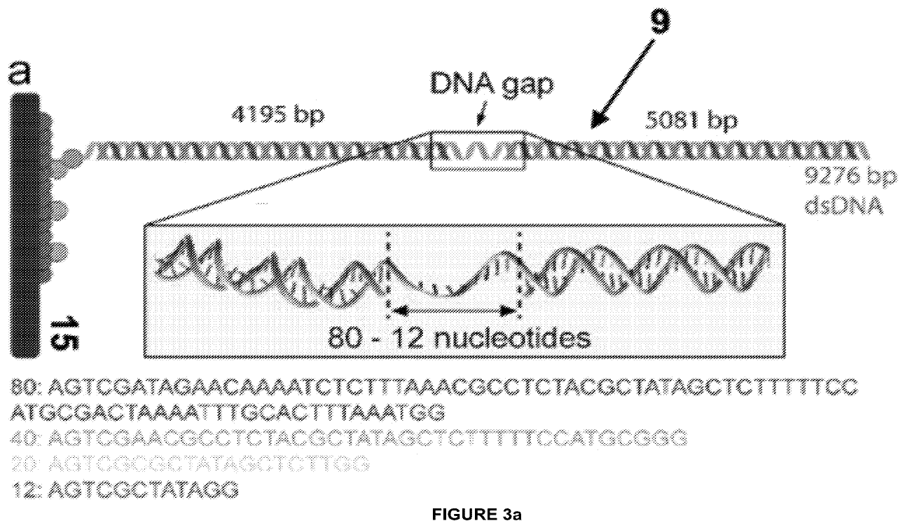

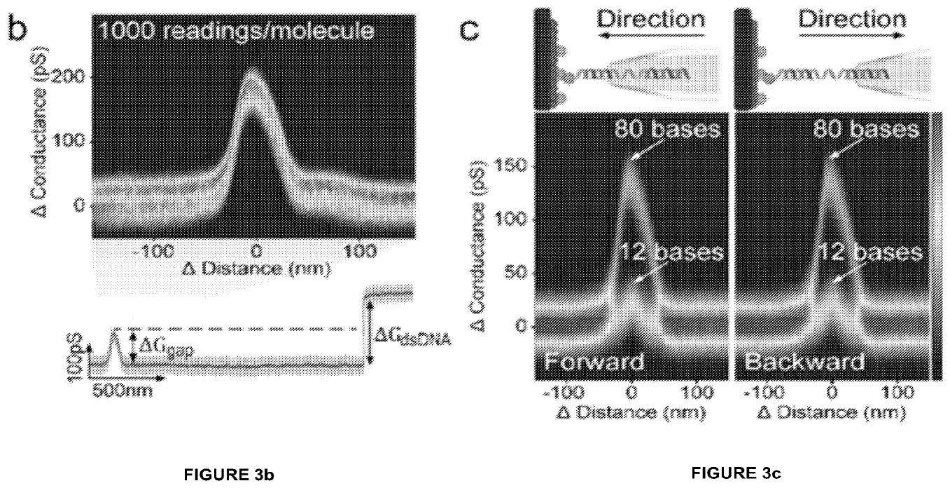

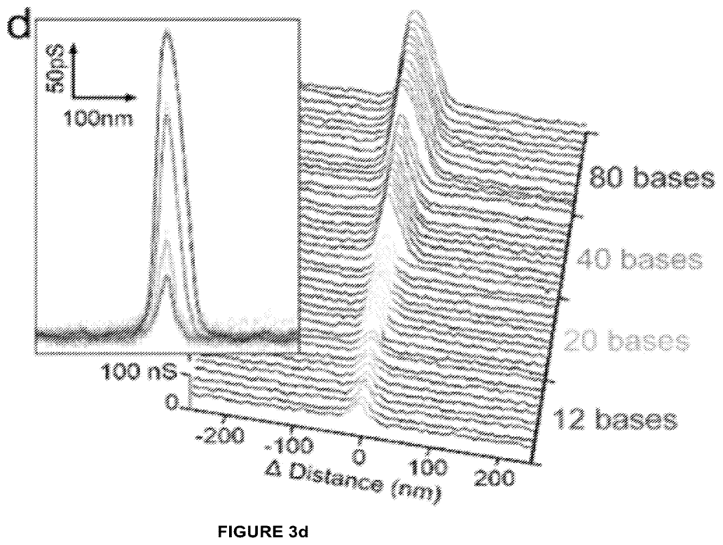

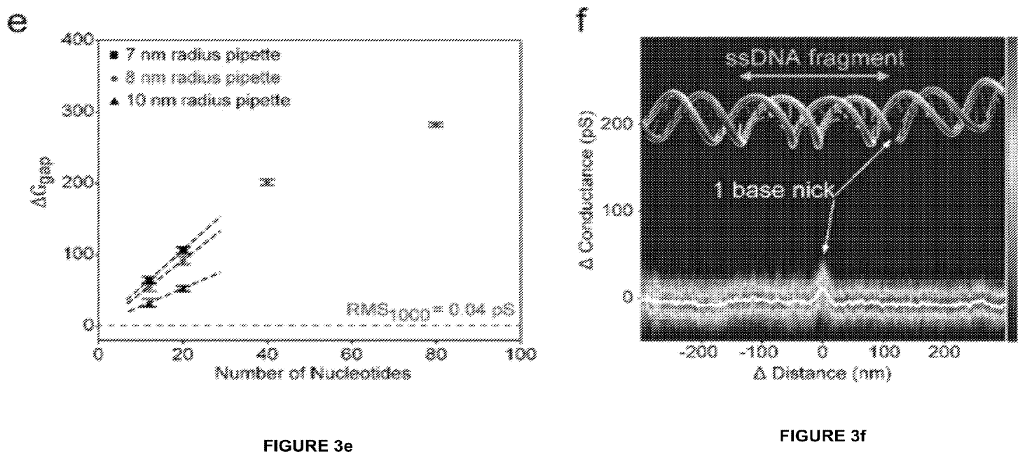

To explore the detection limits of the SICS 1 with glass nanopores 5 as probe 3, the inventors used engineered dsDNA with nucleotide gaps of different sizes (FIG. 3a). Four different DNA constructs were custom-designed to have strands with 9′276 base pairs (2′728 nm). Each construct contained different nucleotide gaps of 80-, 40-, 20-, and 12-nucleotides, corresponding to a length of 27, 14, 7, and 3 nm, respectively. FIG. 3b bottom panel shows a controlled-translocation with SICS on 80-nucleotides that revealed a conductance amplitude (ΔG_{gap}) corresponding to half of the dsDNA conductance drop (ΔG_{dsDNA}). The method allowed thousands readings in the same region of a selected molecule while keeping constant velocity, independent of the bias. FIG. 3b upper panel shows the probability density map of 1000 readings on the same 80-nucleotide DNA gap. By recording and averaging conductance-distance curves on the same molecules, the conductance baseline RMS decreased from 11.2 pS to 0.4 pS, increasing SNR 20 times compared with a single reading. The DNA gap was detected with a standard error of 0.05 pS for an average amplitude of 157.3 pS, which corresponds to an error in the detected amplitude of 0.0003%.

In addition, the SICS 1 can perform bidirectional translocations generating a forward and a backward conductance-distance curve. The inventors performed bidirectional translocations of 80- and 12-nucleotide gaps, revealing identical translocations signatures independently of the pore motion directionality (FIG. 3c). Thus, the hydrodynamic drag force does not affect SICS with asymmetric dynamics, which is critical for controlled translocation in both directions.

To determine the detection limit of the SICS 1, the same pipette and generated conductance-distance curves were used on the specifically synthesized 80-, 40-, 20-, and 10-nucleotides gaps. FIG. 3d shows the conductance-distance curves of the detected nucleotide gaps with an 8 nm glass nanopore radius and FIG. 3e shows the plot of the conductance amplitudes (ΔG_{gap}) vs the number of nucleotides gap. FIG. 3e shows single-base detection after hybridizing a complimentary 19 bases oligonucleotide on a 20 bases DNA gap.

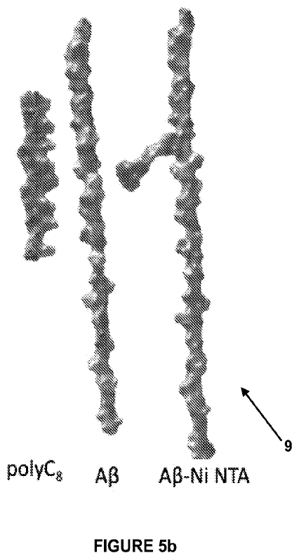

In another exemplary embodiment, SICS 1 has been utilized as a method to monitor post-translational modifications (PTMs) on peptides in situ. Conventional approaches, such as mass spectrometry, primarily operates in a gas phase and may not accurately represent conditions in the condensed phase. In the example (see FIGS. 5a and 5b), a ligand called nitrilotriacetic acid (NTA), that specifically binds to nickel ions, was used. By utilizing SICS 1, it becomes possible to monitor the binding of nickel ions to target peptides. This is due to the spatial addressability of the SICs method so that the same molecules can be measures before and after incubation with nickel ions and one can establish if PTM occurred or not, as well as to localize it.

This allows an operator to investigate and analyze the interaction of the peptides with the specific ions of interest, providing insights into the occurrence and behaviour of PTMs.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments, and be given the broadest reasonable interpretation in accordance with the language of the appended claims.

1.-47. (canceled)

48. Nanopore-based scanning system including:
at least one probe structure comprising at least one nanopore;
a suction device configured to draw an end of at least one (bio)molecule inside the at least one nanopore and inside the at least one probe structure, single or multiple times; and
a displacer configured to mechanically displace the at least one probe structure and the at least one nanopore relative to the at least one (bio)molecule along a direction following a direction of extension of the at least one (bio)molecule while the at least one (bio)molecule is located inside the at least one nanopore and inside the at least one probe structure, or configured to mechanically displace at least one support holding the at least one (bio)molecule relative to the at least one nanopore along a direction following a direction of extension of the at least one (bio)molecule while holding the at least one support and while the at least one (bio)molecule is located inside the at least one nanopore.

49. Nanopore-based scanning system according to claim 48, wherein the displacer is configured to mechanically displace the at least one probe structure and the at least one nanopore in a forward and reverse direction along the direction of extension of the at least one (bio)molecule while the at least one (bio)molecule is located inside the at least one nanopore and inside the at least one probe structure.

50. Nanopore-based scanning system according to claim 48, wherein the displacer includes a mobile element and a fastener configured to attach the probe structure to the mobile element, the mobile element being mobile to displace the probe structure and the nanopore relative to the support and the at least one (bio)molecule to permit controlled and deterministic displacement of the probe structure and the nanopore relative to the support and/or the at least one (bio)molecule.

51. Nanopore-based scanning system according to claim 48, wherein the displacer is configured to mechanically displace the at least one support in a forward and reverse direction along the direction of extension of the at least one (bio)molecule.

52. Nanopore-based scanning system according to claim 48, wherein the displacer includes a mobile element and a fastener configured to attach or hold the support to the mobile element, the mobile element being mobile to displace the support relative to the probe structure and the nanopore to permit controlled and deterministic displacement of the support and the at least one (bio)molecule relative to the probe structure and the nanopore.

53. Nanopore-based scanning system according to claim 48, further including at least one electrode or a first and second electrode arranged to provide an ionic-current measurement during displacement of the at least one probe structure relative to the at least one (bio)molecule, or during displacement of the at least one probe structure relative to the at least one support containing the at least one (bio)molecule.

54. Nanopore-based scanning system according to claim 48, wherein the displacer is configured to displace the at least one probe structure relative to at least one support configured to be functionalized to attach the at least one (bio)molecule thereto, the displacer being configured to mechanically displace the at least one probe structure and/or the at least one support in a least one or a plurality of directions.

55. Nanopore-based scanning system according to claim 48, wherein the suction device comprises an electrophoretic force generator configured to generate an electrophoretic force to draw or suck an end of at least one (bio)molecule inside the at least one nanopore and inside the at least one probe structure.

56. Nanopore-based scanning system according to claim 48, wherein the system is configured to carry out a controlled translocation by drawing the at least one (bio)molecule inside the at least one nanopore and inside the at least one probe structure, and mechanically displacing the at least one probe structure and the at least one nanopore forward and/or backwards along a direction of extension of the at least one (bio)molecule while the at least one (bio)molecule is located inside the at least one nanopore and inside the at least one probe structure.

57. Nanopore-based scanning system according to claim 48, wherein the suction device is configured to draw a free end of at least one tethered or attached (bio)molecule through the at least one nanopore to capture the at least one tethered (bio)molecule inside the at least one probe structure.

58. Nanopore-based scanning system according to claim 48, wherein the at least one nanopore is located at an extremity of the at least one probe structure.

59. Nanopore-based scanning system according to claim 48, wherein the at least one probe structure comprises or consists of at least one glass capillary probe or pipette, or at least one probe including a biological nanopore, or at least one probe including a solid-state nanopore.

60. Nanopore-based scanning system according to claim 48, wherein the at least one probe structure comprises or consists of at least one glass nanopipette or micropipette.

61. Nanopore-based scanning system according to claim 48, further including the at least one (bio)molecule, and/or the least one support comprising the at least one (bio)molecule tethered or fixed thereto.

62. Nanopore-based scanning system according to claim 48, wherein the at least one (bio)molecule comprises or consists of a polymeric molecule.

63. Nanopore-based scanning system according to claim 48, wherein the at least one (bio)molecule comprises or consists of oligonucleotides, or polynucleotides (nucleic acids), or polypeptides, or oligopeptides, or hybrid nucleic acid/peptide constructs, or fatty acids, or glycans, or combination thereof.

64. Nanopore-based scanning system according to claim 48, comprising at least one or a plurality of arrays of probes structures, and at least one or a plurality of arrays of (bio)molecules located on the at least one support.

65. Nanopore-based scanning system according to claim 48, further including a fluorescence microscope or system configured to perform fluorescence measurements of the at least one (bio)molecule.

66. Nanopore based scanning system according to claim 48, wherein the system is configured to determine and control a distance of the at least one probe structure to the at least one support based on the ionic-current passing through the nanopore.

67. Nanopore-based scanning method including the steps of:
providing the nanopore-based scanning system according to claim 48; and
drawing the at least one (bio)molecule inside the at least one nanopore and inside the at least one probe structure and then mechanically displacing the at least one probe structure and the at least one nanopore forward and/or backwards along a direction of extension of the at least one (bio)molecule while the at least one (bio)molecule is located inside the at least one nanopore and inside the at least one probe structure.
